(19) United States
(12) Patent Application Publication
(10) Pub. No.: US 2026/0160691 A1
(43) Pub. Date: Jun. 11, 2026

(54) DEEP LEARNING-BASED PREDICTION USING SPECTROSCOPY

(71) Applicant: AMGEN INC., Thousand Oaks, CA (US)

(72) Inventors: Hamid Khodabandehlou, Thousand Oaks, CA (US); Tony Y. Wang, Tiverton, RI (US); Aditya Tulsyan, Atlanta, GA (US); Gregory L. Schorner, Thousand Oaks, CA (US)

(21) Appl. No.: 18/704,205

(22) PCT Filed: Oct. 26, 2022
(86) PCT No.: PCT/US2022/047790

§ 371 Date: Apr. 24, 2024

Related U.S. Application Data
(60) Provisional application No. 63/272,595, filed on Oct. 27, 2021.

(51) Int. Cl. G01N 21/65; G01N 33/483

(52) U.S. Cl. CPC G01N 21/65; G01N 33/4833

(57) ABSTRACT

A method for monitoring and/or controlling a pharmaceutical process includes obtaining one-dimensional spectral data generated by a spectroscopy system (e.g., a Raman spectroscopy system), converting the one-dimensional spectral data to a two-dimensional spectral data matrix, and applying the two-dimensional spectral data matrix to an input layer of a deep learning model (e.g., a convolutional neural network). The deep learning model predicts a parameter (e.g., metabolite level) based on the two-dimensional spectral data matrix, e.g., in order to monitor and/or control a pharmaceutical process.

### FIELD OF THE DISCLOSURE

The present application relates generally to the monitoring and/or control of pharmaceutical (e.g., biopharmaceutical) processes using spectroscopic techniques (e.g., Raman spectroscopy), and more specifically relates to the use of deep learning in connection with such spectroscopic techniques.

### BACKGROUND

Stable production of biotherapeutic proteins by a biopharmaceutical process generally requires that a bioreactor maintain balanced and consistent parameters (e.g., cellular metabolic concentrations), which in turn demands rigorous process monitoring and control. To meet these demands, process analytical technology (PAT) tools are increasingly being adopted. Online monitoring of pH, dissolved oxygen, and cell culture temperature are a few examples of traditional PAT tools that have been used in feedback control systems. In recent years, other in-process probes have been investigated and deployed for continuous monitoring of more complex species, such as viable cell density (VCD), glucose, lactate, and other critical cellular metabolites, amino acids, titer, and critical quality attributes.

In biopharmaceutical and other (e.g., small molecule) areas, advanced process control techniques typically rely on real-time and frequent measurements from the process under control. However, such measurements may be unavailable or burdensome. In the biopharmaceutical industry, for example, real-time measurements are often not available and instead the scientists rely on offline samples (e.g., taken once a day) to monitor the bioprocess. Increasing the number of offline samples to have a more holistic view of the process may not be feasible due to working volume constraints of the size of the bioreactor or resource limitations, for example.

To enable real-time trending of bioprocess cultures, tools such as Raman spectroscopy are often used. In this setup, in-situ Raman probes are inserted into the bioreactors to collect Raman spectra. Raman spectroscopy is a popular PAT tool widely used for online monitoring in biomanufacturing. It is an optical method that enables non-destructive analysis of chemical composition and molecular structure. In Raman spectroscopy, incident laser light is scattered inelastically due to molecular vibration modes. The frequency difference between the incident and scattered photons is referred to as the “Raman shift,” and the vector of Raman shift (usually expressed in terms of wave number) versus intensity levels (referred to herein as a “Raman spectrum,” a “Raman scan,” or a “Raman scan vector”) can be analyzed to determine the chemical composition and molecular structure of a sample. Applications of Raman spectroscopy in polymer, pharmaceutical, biomanufacturing and biomedical analysis have surged in the past three decades as laser sampling and detector technology have improved. Due to these technological advances, Raman spectroscopy is now a practical analysis technique used both within and outside of the laboratory. Since the application of in-situ Raman measurements in biomanufacturing was first reported, it has been adopted to provide online, real-time predictions of several key process states, such as glucose, lactate, glutamate, glutamine, ammonia, VCD, and so on. These predictions are typically based on a calibration model or soft-sensor model that is built in an offline setting, based on analytical measurements from an analytical instrument. Partial least squares (PLS) and multiple linear regression modeling methods are commonly used to correlate the Raman spectra to the analytical measurements. These models typically require pre-processing filtering of the Raman scans prior to calibrating against the analytical measurements. Once a calibration model is trained, the model is implemented in a real-time setting to provide in-situ measurements for process monitoring and/or control.

Raman model calibration for biopharmaceutical applications is nontrivial, as biopharmaceutical processes typically operate under stringent constraints and regulations. The current state-of-the-art approach for Raman model calibration in the biopharmaceutical industry is to first run multiple campaign trials to generate relevant data that is used to correlate the Raman spectra to the analytical measurement(s). These trials are both expensive and time-consuming, as each campaign may last between two to four weeks in a laboratory setting, for example. Further, only limited samples may be available for the analytical instruments (e.g., to ensure that a lab-scale bioreactor maintains a healthy mass of viable cells). In fact, it is not uncommon to have only one or two measurements available each day from in-line or offline analytical instruments. To further exacerbate the situation, the current best practices yield calibration models that are tied to a specific process, the specific formula or profile of the bioreactor media, and the specific operating conditions. Thus, if any of the aforementioned variables were to change, the models may need to be recalibrated based on new data. In fact, both Raman model calibration and model maintenance require significant resource allocations and are typically performed in an offline setting. While approaches that adapt models to new operating conditions have been proposed (e.g., recursive, moving-window, and time-difference methods), these methods may be unable to adequately handle abrupt process changes.

There are a number of publications describing generic Raman models based on traditional chemometric methods (e.g., PLS modeling) for multiple molecules. However, these generic models assume that the processes use similar, if not the same, media formulations and/or process conditions. Under these models, the media and processes are usually platformed with little or no variation. The drawback of this type of generic model is that once a process deviates from the norm, or if the training data set contains too wide of a process range in an effort to account for the variations (e.g., media additives, process duration and/or other process changes) between the different molecules, the generic models lose accuracy and precision. Therefore, these “generic” models are only generic within the described strict boundaries. See Mehdizaheh et al., Biotechnol. Prog. 31 (4): 1004-1013, 2015; Webster et al., Biotechnol. Prog. 34 (3): 730-737, 2018.

More recently, a system employing automatic calibration and automatic maintenance of Raman spectroscopic models using Just in Time Learning (JITL) for real-time predictions has been described. See International Patent Publication No. WO2020/086635. When used in isolation, however, JITL typically requires ongoing (though less frequent) analytical measurements for recalibration, which may not be feasible (e.g., in small bioreactors), consumes time and other resources, and can provide different results when measurements are rerun. On the other hand, if recalibration is not performed (e.g., if “offline” JITL is used), results can vary greatly depending on modality and the amount and type of historical data available.

### BRIEF SUMMARY

The term “pharmaceutical process” refers to a process used in pharmaceutical manufacturing and/or development, such as a cell culture process to produce a desired recombinant protein or a small molecule manufacturing process. In the biopharmaceutical context, cell culture takes place in a cell culture vessel, such as a bioreactor, under conditions that support the growth and maintenance of an organism engineered to express the protein. During recombinant protein production, process parameters, such as media component concentrations, including nutrients and metabolites (e.g., glucose, lactate, glutamate, glutamine, ammonia, amino acids, Na+, K+ and other nutrients or metabolites), media state (pH, pCO₂, pO₂, temperature, osmolality, etc.), as well as cell and/or protein parameters (e.g., viable cell density (VCD), titer, cell state, critical quality attributes, etc.) are monitored for control and/or maintenance of the cell culture process.

To address some of the aforementioned limitations of the current best industrial practices, embodiments described herein relate to systems and methods that improve upon traditional techniques for spectroscopic analysis of pharmaceutical processes, such as Raman spectroscopy. In particular, deep learning models such as convolutional neural networks (CNNs) are used as an alternative modeling method to predict process-related parameters such as metabolite concentrations. It is understood that the term “predicting” (or “predicts,” “prediction,” etc.) is used broadly herein to refer to predicting and/or inferencing. CNNs are feedforward neural networks specialized for processing images, e.g., to perform object detection and classification. However, Raman and other (e.g., NIR, HPLC, etc.) spectroscopic measurements are not images, and thus are not natural candidates for CNN processing. Nonetheless, the systems and methods described herein generate “pseudo-images” from spectroscopic scans, and process those pseudo-images using one or more CNNs (e.g., one CNN per metabolite or other process parameter of interest, etc.). Deep CNN(s) and Raman spectroscopic measurements can be used to create an offline model, which may be product-agnostic, and which predicts one or more parameters or characteristics of a pharmaceutical process (e.g., a product quality attribute). This can allow the use of the model on different processes without the need for recalibration or retraining. Another advantage of CNNs is their weight sharing feature. This weight sharing feature of CNNs enables their parameter number to be reduced substantially compared to traditional deep neural networks. Additionally, this allows CNN models to be trained using smaller training data sets.

The deep CNN is a general offline model which can be used to predict metabolite concentrations using spectroscopic measurements from any process and can be finely tuned to a specific process for optimized performance. The model does not require a priori knowledge of the process and thus is a true generic spectroscopy modeling solution for all processes. The deep learning CNN approach overcomes many of the problems associated with chemometric methods, such as the need for frequent analytical measurements, the inability to frequently measure in small bioreactors, the time delay between sampling and obtaining the measurement, and the potential for lack of reproducibility when rerunning a measurement.

In contrast to a JITL platform, which maintains a dynamic library that is typically updated each time a new analytical measurement is available, a CNN approach does not necessarily update the model each time a new analytical measurement is taken. Instead, the input scan is fed to a CNN model which was previously generated/trained. With the CNN approach, the CNN model can optionally be updated after the prediction or process control takes place.

In contrast to Gaussian process models which generally do not require pre-processing filtering of the spectral data (e.g., Raman scans), the CNN models use pre-processing of the Raman scans.

The deep learning (e.g., CNN) approach described here can be used in conjunction with JITL/PLS or other techniques for process monitoring and control, or independently of such techniques.

### BRIEF DESCRIPTION OF THE DRAWINGS

The skilled artisan will understand that the figures, described herein, are included for purposes of illustration and are not limiting on the present disclosure. The drawings are not necessarily to scale, emphasis instead being placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 is a simplified block diagram of an example system that may be used to for process monitoring.

FIG. 2 is a simplified block diagram of an example system that may be used to for closed-loop control of glucose concentration.

FIG. 3 depicts a representative convolutional neural network (CNN).

FIG. 4 depicts an example data flow that may occur in the system of FIG. 1 to enable and perform analysis of pharmaceutical processes using a deep learning model.

FIG. 5 depicts example pre-processing of spectral data that may be implemented in the system of FIG. 1.

FIG. 6 depicts another example data flow that may occur in the system of FIG. 1 when analyzing a pharmaceutical process using a deep learning model.

FIG. 7 is a flow diagram of an example method for using techniques of the present disclosure in combination with Just In Time Learning (JITL).

FIG. 8 depicts experimental results for prediction of VCD using deep learning and pre-processing techniques described herein.

FIG. 9 depict experimental results for prediction of viability using deep learning and pre-processing techniques described herein.

FIG. 10 depicts experimental results for prediction of TCD using deep learning and pre-processing techniques described herein.

FIG. 11 depicts experimental results for prediction of glucose using deep learning and pre-processing techniques described herein.

FIG. 12 depicts experimental results for prediction of lactate using deep learning and pre-processing techniques described herein.

FIG. 13 depicts experimental results for prediction of osmolality using deep learning and pre-processing techniques described herein.

FIG. 14 depicts experimental results for prediction of glutamate using deep learning and pre-processing techniques described herein.

FIG. 15 depicts experimental results for prediction of glutamine using deep learning and pre-processing techniques described herein.

FIG. 16 depicts experimental results for prediction of potassium using deep learning and pre-processing techniques described herein.

FIG. 17 depicts experimental results for prediction of sodium using deep learning and pre-processing techniques described herein.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

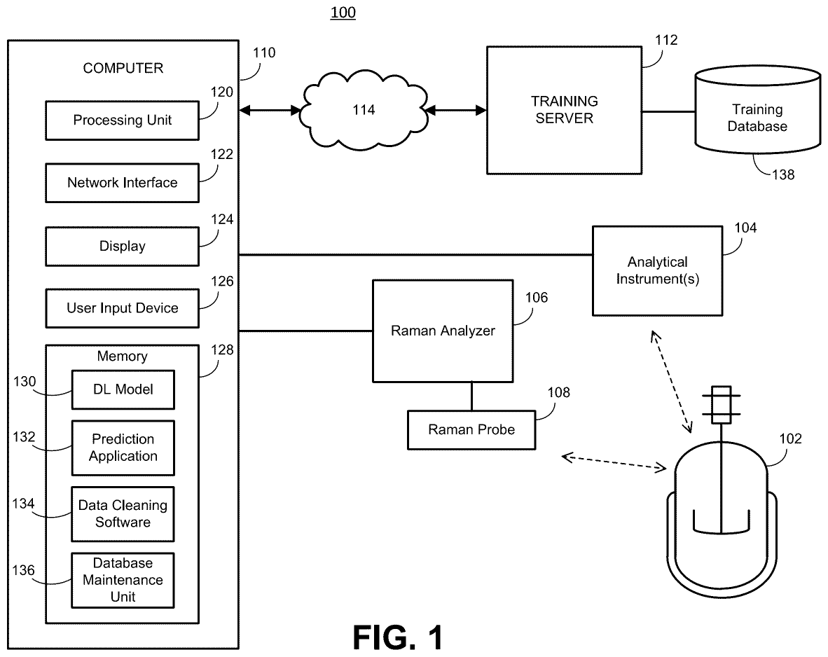

FIG. 1 is a simplified block diagram of an example system 100 that may be used to predict parameters or characteristics of biopharmaceutical processes. While FIG. 1 depicts a system 100 that implements Raman spectroscopy techniques for a biopharmaceutical process, it is understood that, in other embodiments, system 100 may implement other suitable spectroscopy techniques (e.g., near-infrared (NIR) spectroscopy, high performance liquid chromatography (HPLC), ultra high performance liquid chromatography (UPLC) spectroscopy, mass spectrometry, etc.), and/or may implement such techniques with respect to non-biopharmaceutical processes (e.g., small molecule pharmaceutical processes).

System 100 includes a bioreactor 102, one or more analytical instruments 104, a Raman analyzer 106 with Raman probe 108, a computer 110, and a training server 112 that is coupled to computer 110 via a network 114. Bioreactor 102 may be any suitable vessel, device or system that supports a biologically active environment, which may include living organisms and/or substances derived therefrom (e.g., a cell culture) within a media. Bioreactor 102 may contain recombinant proteins that are being expressed by the cell culture, e.g., such as for research purposes, clinical use, commercial sale or other distribution. Depending on the biopharmaceutical process being monitored, the media may include a particular fluid (e.g., a “broth”) and specific nutrients, and may have target media state parameters, such as a target pH level or range, a target temperature or temperature range, and so on. The media may also include organisms and substances derived from the organisms such as metabolites and recombinant proteins. Collectively, the contents and parameters/characteristics of media are referred to herein as the “media profile.”

Analytical instrument(s) 104 may be any in-line, at-line and/or offline instrument, or instruments, configured to measure one or more characteristics or parameters of the biologically active contents within bioreactor 102, based on samples taken therefrom. For example, analytical instrument(s) 104 may measure one or more media component concentrations, such as nutrient and/or metabolite levels (e.g., glucose, lactate, glutamate, glutamine, ammonia, amino acids, Na+, K+, etc.) and media state parameters (pH, pCO₂, pO₂, temperature, osmolality, etc.). Additionally, or alternatively, analytical instrument(s) 104 may measure osmolality, viable cell density (VCD), titer, critical quality attributes, cell state (e.g., cell cycle) and/or other characteristics or parameters associated with the contents of bioreactor 102. As a more specific example, samples may be taken, spun down, purified by one or more columns, and run through a first one of analytical instruments 104 (e.g., an HPLC or UPLC instrument), followed by a second one of analytical instruments 104 (e.g., a mass spectrometer), with both the first and second analytical instruments 104 providing analytical measurements. One, some or all of analytical instrument(s) 104 may use destructive analysis techniques.

Raman analyzer 106 may include a spectrograph device coupled to Raman probe 108 (or, in some implementations, multiple Raman probes). Raman analyzer 106 may include a laser light source that delivers the laser light to Raman probe 108 via a fiber optic cable, and may also include a charge-coupled device (CCD) or other suitable camera/recording device to record signals that are received from Raman probe 108 via another channel of the fiber optic cable, for example. Alternatively, the laser light source may be integrated within Raman probe 108 itself. Raman probe 108 may be an immersion probe, or any other suitable type of probe (e.g., a reflectance probe and transmission probe).

Collectively, Raman analyzer 106 and Raman probe 108 form a Raman spectroscopy system that is configured to non-destructively scan the biologically active contents during the biopharmaceutical process within bioreactor 102 by exciting, observing, and recording a molecular “fingerprint” of the biopharmaceutical process. The molecular fingerprint corresponds to the vibrational, rotational and/or other low-frequency modes of molecules within the biologically active contents within the biopharmaceutical process when the bioreactor contents are excited by the laser light delivered by Raman probe 108. As a result of this scanning process, Raman analyzer 106 generates one or more Raman scan vectors that each represent intensity as a function of Raman shift (a frequency-related parameter). A Raman scan vector may be intensity values as a function of wave number (e.g., in units of cm⁻¹), for example.

More generally, the system 100 may include any spectroscopy system (e.g., Raman spectroscopy system, NIR spectroscopy system, HPLC spectroscopy system, etc.) that generates 1D spectral data. As used herein, “1D spectral data” refers to values of spectral data (e.g., intensity values) that are not arranged in a matrix format with two or more dimensions. For example, 1D spectral data may be a string/sequence of tuples each having the format [wave number, intensity value]. As another example, 1D spectral data may simply be a string/sequence of intensity values, so long as the order of the intensity values within the string is in accordance with a known/predetermined format (e.g., with each position within the string corresponding to a respective wave number). In some embodiments, the 1D spectral data may be expressed as a function of a spectral parameter other than wave number (e.g., wavelength or frequency).

Computer 110 is coupled to Raman analyzer 106 and analytical instrument(s) 104, and is generally configured to analyze the Raman scan vectors generated by Raman analyzer 106 in order to predict one or more characteristics or parameters of the biopharmaceutical process. For example, computer 110 may analyze the Raman scan vectors to predict the same type(s) of characteristics or parameters that are measured by analytical instrument(s) 104. As a more specific example, computer 110 may predict glucose concentrations, while analytical instrument(s) 104 actually measure glucose concentrations. However, whereas analytical instrument(s) 104 may make relatively infrequent, “offline” analytical measurements of samples extracted from bioreactor 102 (e.g., due to limited quantities of the media from the biopharmaceutical process, and/or due to the higher cost of making such measurements, etc.), computer 110 may make relatively frequent, “online” predictions of characteristics or parameters in real-time. Computer 110 may also be configured to transmit analytical measurements made by analytical instrument(s) 104 to training server 112 via network 114, as will be discussed in further detail below.

In the example embodiment shown in FIG. 1, computer 110 includes a processing unit 120, a network interface 122, a display 124, a user input device 126, and a memory 128. Processing unit 120 includes one or more processors, each of which may be a programmable microprocessor that executes software instructions stored in memory 128 to execute some or all of the functions of computer 110 as described herein. Alternatively, one or more of the processors in processing unit 120 may be other types of processors (e.g., application-specific integrated circuits (ASICs), field-programmable gate arrays (FPGAs), etc.). Memory 128 may include one or more physical memory devices or units containing volatile and/or non-volatile memory. Any suitable memory type or types may be used, such as read-only memory (ROM), solid-state drives (SSDs), hard disk drives (HDDs), and so on.

Network interface 122 may include any suitable hardware (e.g., front-end transmitter and receiver hardware), firmware, and/or software configured to communicate via network 114 using one or more communication protocols. For example, network interface 122 may be or include an Ethernet interface. Network 114 may be a single communication network, or may include multiple communication networks of one or more types (e.g., one or more wired and/or wireless local area networks (LANs), and/or one or more wired and/or wireless wide area networks (WANs) such as the Internet or an intranet, for example).

Display 124 may use any suitable display technology (e.g., LED, OLED, LCD, etc.) to present information to a user, and user input device 126 may be a keyboard or other suitable input device. In some embodiments, display 124 and user input device 126 are integrated within a single device (e.g., a touchscreen display). Generally, display 124 and user input device 126 may combine to enable a user to interact with graphical user interfaces (GUIs) provided by computer 110, e.g., for purposes such as manually monitoring various processes being executed within system 100. In some embodiments, however, computer 110 does not include display 124 and/or user input device 126, or one or both of display 124 and user input device 126 are included in another computer or system that is communicatively coupled to computer 110 (e.g., in some embodiments where predictions are sent directly to a control system that implements closed-loop control).

Memory 128 stores the instructions of one or more software applications and data used by and/or output by such applications, and possibly other data or data structures. In the example of FIG. 1, memory 128 stores at least a deep learning (DL) model 130, a prediction application 132, data cleaning software 134, and a database maintenance unit 136. Prediction application 132, when executed by processing unit 120, is generally configured to use DL model 130 to predict parameters of the biopharmaceutical process in bioreactor 102 (e.g., parameters of the sort that can be measured by the analytical instrument(s) 104) by processing Raman scan vectors generated by Raman analyzer 106. Depending on the frequency at which Raman analyzer 106 generates such scan vectors, the prediction application 132 may predict characteristics or parameters on a periodic or other suitable time basis. Raman analyzer 106 may itself control when scan vectors are generated, or computer 110 may trigger the generation of scan vectors by sending a command to Raman analyzer 106, for example. The prediction application 132 may use a single DL model 130 to predict only a single type of characteristic or parameter based on each scan vector (e.g., only glucose concentration), or may use multiple DL models to predict multiple types of characteristics or parameters based on each scan vector (e.g., glucose concentration and viable cell density). Prediction application 132 and DL model 130 will be discussed in further detail below.

Data cleaning software 134 generally removes noise and/or outliers from the scan vectors or otherwise optimizes the scan vectors generated by Raman analyzer 106, prior to processing by prediction application 132. Database maintenance unit 136 generally updates training data in a training database 138 by sending training server 112 new Raman scan vectors and corresponding analytical measurements made by analytical instrument(s) 104. In some embodiments, however, data cleaning software 134 and/or database maintenance unit 136 are not included in system 100.

Training server 112 may be remote from computer 110 (e.g., such that a local setup may include only bioreactor 102, analytical instrument(s) 104, Raman analyzer 106 with Raman probe 108, and computer 110) and, as seen in FIG. 1, may contain or be communicatively coupled to a training database 138 that stores observation data sets associated with past observations. Each observation data set in training database 138 may include spectral data (e.g., one or more Raman scan vectors of the sort produced by Raman analyzer 106, or other 1D spectral data produced by a different type of spectroscopy system) and one or more corresponding analytical measurements (e.g., one or more measurements of the sort(s) produced by analytical instrument(s) 104). Depending on the embodiment and/or scenario, the past observations may have been collected for a number of different biopharmaceutical processes, under a number of different operating conditions (e.g., different metabolite concentration set points), and/or with a number of different media profiles (e.g., different fluids, nutrients, PH levels, temperatures, etc.). Generally, it may be desirable to have training database 138 represent a broadly diverse collection of processes, operating conditions, and media profiles. Training database 138 may or may not store information indicative of those processes, cell lines, proteins, metabolites, operating conditions, and/or media profiles, however, depending on the embodiment. In some embodiments, training server 112 is remotely coupled to multiple other computers similar to computer 110, via network 114 and/or other networks. This may be desirable in order to collect a larger number of observation data sets for storage in training database 138.

Training server 112 trains DL model 130. That is, training server 112 uses historical Raman scan vector(s), and possibly other feature data, associated with each observation data set as a feature set, and uses the analytical measurement(s) associated with the same observation data set as a label for that feature set. Training server 112 then provides DL model 130 to computer 110 via network 114. In other embodiments, server 112 does not provide DL model 130 to computer 110, but instead operates DL model 130 (and possibly prediction application 132 as a whole) as a cloud-based service. For example, server 112 may locally store both prediction application 132 and DL model 130, or may locally store only DL model 130 (in which case the prediction application 132 at computer 110 makes use of DL model 130 via network 114 and any appropriate application programming interface(s)). In still other embodiments, system 100 does not include training server 112, and computer 110 directly accesses training database 138. For example, training database 138 may be stored in memory 128.

It is understood that other configurations and/or components may be used instead of those shown in FIG. 1. For example, a different computer (not shown in FIG. 1) may transmit measurements provided by analytical instrument(s) 104 to training server 112, one or more additional computing devices or systems may act as intermediaries between computer 110 and training server 112, some or all of the functionality of computer 110 as described herein may instead be performed remotely by training server 112 and/or another remote server, and so on. For ease of explanation, the remaining description will assume that training database 138 is coupled to training server 112, as depicted in FIG. 1. However, one of ordinary skill in the art will readily understand how the communication paths may differ if training database 138 were instead local to computer 110, or in another suitable location within a system architecture.

After DL model 130 is trained (e.g., by training server 112), and during run-time operation of system 100, Raman analyzer 106 and Raman probe 108 scan (i.e., generate Raman scan vectors for) a biopharmaceutical process in bioreactor 102, and Raman analyzer 106 transmits the Raman scan vector(s) to computer 110. Raman analyzer 106 and Raman probe 108 may provide scan vectors to support predictions (made by prediction application 132) according to a predetermined schedule of monitoring periods, such as once per minute, or once per hour, etc. Alternatively, predictions may be made at irregular intervals (e.g., in response to a certain process-based trigger, such as a change in measured pH level and/or temperature), such that each monitoring period has a variable or uncertain duration. Depending on the embodiment, Raman analyzer 106 may send only one scan vector to computer 110 per monitoring period, or multiple scan vectors to computer 110 per monitoring period, depending on how many scan vectors DL model 130 accepts as input for a single prediction. Multiple scan vectors (e.g., when aggregated or averaged) may improve the prediction accuracy of DL model 130, for example.

In some embodiments, DL model 130 is not retrained/recalibrated after initial training, or training server 112 only does so infrequently (e.g., relative to traditional techniques or JITL). In other embodiments, however, prediction application 132, another application in memory 128, retrains/recalibrates the local DL model 130 more often using JITL techniques (e.g., any of the techniques discussed in International Patent Publication No. WO2020/086635, which is hereby incorporated herein by reference).

After receiving a Raman scan vector, prediction application 132 pre-processes the scan vector (as discussed further below) to generate a pseudo-image, and applies the pseudo-image as an input to DL model 130. DL model 130 then generates a prediction based on the pseudo-image. In some embodiments, DL model 130 also accepts other information as part of the input/feature set (e.g., operating conditions, media profile, process data, cell line information, protein information, metabolite information, etc.).

Database maintenance unit 136 may cause analytical instrument(s) 104 to periodically collect one or more actual analytical measurements, at a significantly lower frequency than the monitoring period of Raman analyzer 106 (e.g., only once or twice per day, etc.). The measurement(s) by analytical instrument(s) 104 may be destructive, in some embodiments, and require permanently removing a sample from the process in bioreactor 102. At or near the time that database maintenance unit 136 causes analytical instrument(s) 104 to collect and provide the actual analytical measurement(s), database maintenance unit 136 may also cause Raman analyzer 106 to provide one or more Raman scan vectors. Database maintenance unit 136 may then cause network interface 122 to send the Raman scan vector(s) and corresponding actual analytical measurement(s) to training server 112 via network 114, for storage as a new observation data set in training database 138. Training database 138 may be updated according to any suitable timing, which may vary depending on the embodiment. If analytical instrument(s) 104 output(s) actual analytical measurements within seconds of measuring a sample, for instance, training database 138 may be updated with new measurements almost immediately as samples are taken. In certain other embodiments, however, the actual analytical measurements may be the result of minutes, hours, or even days of processing by one or more of analytical instrument(s) 104, in which case training database 138 is not updated until after such processing has been completed. In still other embodiments, new observation data sets may be added to training database 138 in an incremental manner, as different ones of analytical instruments 104 complete their respective measurements. In any of these embodiments, training database 138 may provide a “dynamic library” of past observations that training server 112 may draw upon for tuning or retraining DL model 130. In other embodiments, however, database maintenance unit 136 is omitted, training database 138 is not updated, and/or DL model 130 is not tuned or retrained.

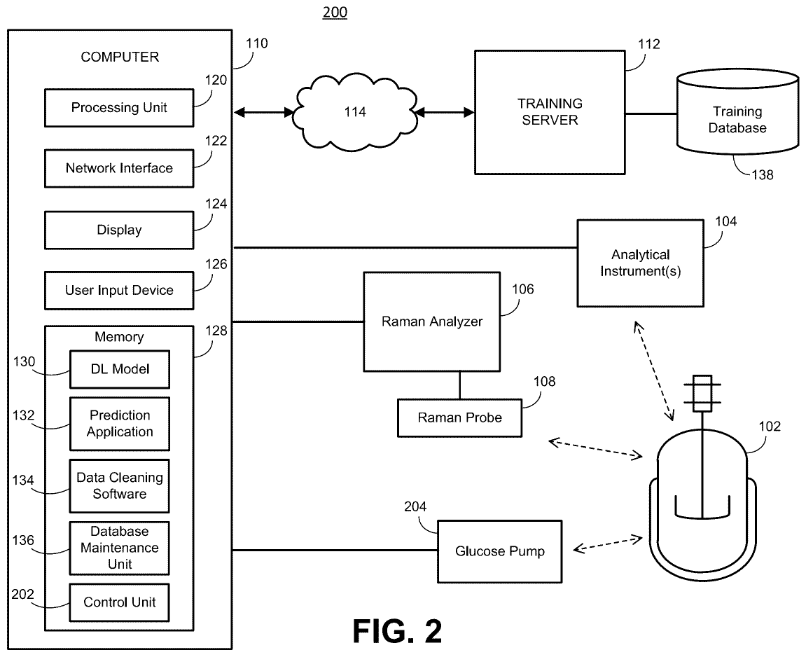

Prediction application 132 may predict the parameter(s) for various purposes, depending on the embodiment and/or scenario. For example, certain parameters may be monitored (i.e., predicted) as a part of a quality control process, to ensure that the process still complies with relevant specifications. As another example, one or more parameters may be monitored/predicted to provide feedback in a closed-loop control system. For example, FIG. 2 depicts a system 200 that is similar to system 100, but controls a glucose concentration in the biopharmaceutical process (i.e., adds additional glucose to the predicted glucose concentration to match a desired set point, within some acceptable tolerance). It is understood that, in other embodiments, system 200 may instead (or also) be used to control process parameters other than glucose level, or to control glucose level based on predictions of one or more other process parameters (e.g., lactate level, pH, etc.). In FIG. 2, the same reference numbers are used to indicate the corresponding components from FIG. 1.

As seen in FIG. 2, within system 200, memory 128 additionally stores a control unit 202. Control unit 202 is configured to control a glucose pump 204, i.e., to cause glucose pump 204 to selectively introduce additional glucose into the biopharmaceutical process within bioreactor 102. Control unit 202 may comprise software instructions that are executed by processing unit 120, for example, and/or appropriate firmware and/or hardware. In some embodiments, control unit 202 implements a model predictive control (MPC) technique, using glucose concentrations as inputs in a closed-loop architecture. In embodiments where DL model 130 provides credibility bounds or other confidence indicators with each prediction, control unit 202 may also accept the confidence indicators as inputs. For example, control unit 202 may only generate control instructions for glucose pump 204 based on glucose concentration predictions having a sufficiently high confidence indicator (e.g., only based on predictions associated with credibility bounds that do not exceed some percentage or absolute measurement range, or only based on predictions associated with confidence scores over some minimum threshold score, etc.), or may increase and/or reduce the weight of a given prediction based on its confidence indicator, etc.

As discussed further below, prediction application 132 converts 1D spectral data (e.g., Raman scan vectors) into an image-like format, which is a 2D matrix of values (also referred to herein as a “pseudo-image”). For example, if the 1D spectral data is a sequence of at least j×k values (e.g., an array of intensity values in which each position corresponds to a different wave number, or a sequence of [wave number, intensity value] tuples, etc.), prediction application 132 may convert the sequence into a 2D spectral data matrix with j rows and k columns, with each position in the 2D spectral data matrix corresponding to a different wave number. In particular, prediction application 132 may place the first N (>1) intensity values (or [wave number, intensity value] tuples) of the sequence into Row 1 (or Column 1) of the matrix, place the second N intensity values (or [wave number, intensity value] tuples) of the sequence into Row 2 (or Column 2) of the matrix, and so on.

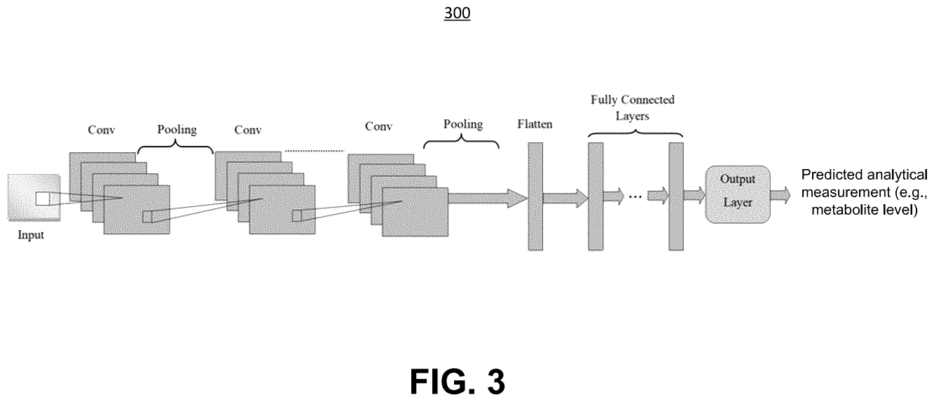

DL model 130 of FIG. 1 or FIG. 2 may be any deep learning model that is configured to process image data, and is therefore capable of processing such pseudo-images. In some embodiments, DL model 130 is (or includes) a convolutional neural network (CNN), which is a feedforward neural network specialized for processing images. An example CNN 300 that may be used as DL model 130 (or a portion thereof) is shown in FIG. 3. CNN 300 includes an input layer, a number of convolution layers, a number of pooling layers, a flatten layer, a number of fully connected (dense) layers, and an output layer. Prediction application 132 applies the pseudo-image (2D spectral data matrix) to the input layer, which is a passive layer that passes the pseudo-image to the first convolution layer. The convolution layer(s) apply multiple filters to the pseudo-image through convolution operations, and extract features from the pseudo-image. The convolution operation may be defined by the following equation:

$$G [ m , n ] = ( f * h ) [ m , n ] = {\sum }_{j \vert a} {\sum }_{k \vert b} h [ j , k ] f [ m - j , n - k ] \qquad ( Equation 1 )$$

In Equation 1, f is the input (pseudo-image), h is the filter or kernel, m and n are result matrix rows and column indices (respectively), and a and b are stride parameters (which may be assumed to be 1 in CNN 300).

The output of the convolution layer(s) may be fed to an activation function. Although CNN 300 may implement activation functions such as sigmoid, tangent hyperbolic (tanh), and/or linear functions, rectified linear units (Relu) may be used instead in order to avoid vanishing gradient issues. The Relu activation function may be defined as:

$$g ( x ) = Relu ( x ) = max ( 0 , x ) \qquad ( Equation 2 )$$

The tangent hyperbolic function may be defined as:

$$g ( x ) = tanh ( x ) = \frac{{e}^{x} - {e}^{- x}}{{e}^{x} + {e}^{- x}} \qquad ( Equation 3 )$$

CNN 300 may include the pooling layers after each of (or each of some of) the convolution layers. Each pooling layer applies the pooling operation to the output of the preceding convolution layer. The pooling operation may be a maximum, average, minimum, or other statistical measure of the feature map. The pooling layers increase the computational efficiency of CNN 300 by reducing the size of the convolution output while generally preserving the most relevant information. In some embodiments, CNN 300 includes max pooling and average pooling layers.

The flatten layer of CNN 300 may follow the last convolution layer, or follow the last pooling layer (e.g., if the last convolution layer is followed by a pooling layer). The flatten layer transforms the output of the last convolution or pooling layer into a vector, which is then fed to a fully connected, linear, and/or softmax layer. The fully connected layers of CNN 300 may follow the convolution and pooling layers. Fully connected layers are similar to the internal layers of shallow neural networks, and perform high-level reasoning from the output of the convolutional and pooling layers. The output layer of CNN 300 may perform image classification applications and therefore may be a softmax layer to determine the class of the input pseudo-image. Because CNN 300 solves a regression problem, CNN 300 may include a fully connected layer with a linear activation function as the output layer.

As described above, CNN 300 may include multiple convolution, pooling, and dense layers (e.g., the activation function of the convolution and dense layers may be linear, tangent hyperbolic or rectified linear units, and average pooling and max pooling layers may be used). Once CNN 300 is developed, various techniques can be used to optimize the model. In one embodiment, a cost function is employed. The cost function can be selected from mean absolute percentage error and mean squared error, for example. In one embodiment, an optimization algorithm is employed. CNN 300 can be optimized using any suitable optimization algorithm to learn the relationship between the Raman (or other) spectra and the desired metabolite level (or other predicted characteristic), such as stochastic gradient descent, root mean square propagation (RMSProp), Adamax, Adagrad, Adadelta, and so on. Once optimization is finished, CNN 300 can be tested against different data sets to evaluate/validate the model performance. If the model performance is not satisfactory, the number of layers, the activation functions, and/or the optimization algorithm can be modified to achieve better model performance.

In some embodiments, DL model 130 includes multiple deep learning models (e.g., multiple CNNs similar to CNN 300), each of which is trained and/or optimized to predict a different type of parameter. For example, the prediction application 132 may apply a given Raman scan vector to a first CNN to predict a glucose concentration, to a second CNN to predict a lactate concentration, to a third CNN to predict osmolality, and so on. The various CNNs may be developed using different numbers of layers and/or nodes, different activation functions, different training and/or optimization algorithms, different loss functions, and so on.

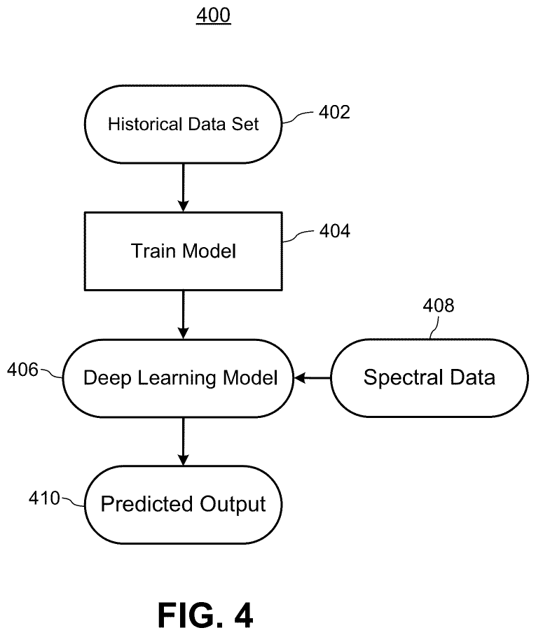

FIG. 4 is an example data flow 400 that may occur in system 100 of FIG. 1 to enable and perform analysis of pharmaceutical processes using a deep learning model such as DL model 130 (e.g., CNN 300). In data flow 400, a historical data set 402 may reside in training database 138. The historical data set 402 includes spectral data (e.g., Raman scan vectors or other 1D spectral data) generated by suitable devices/systems (e.g., similar to Raman analyzer 106 and Raman probe 108, or a different type of spectroscopy system), and corresponding labels. The labels may be actual measurements of the parameter (e.g., metabolite level) of interest, taken by an analytical instrument (similar to instrument(s) 104) at the same time, or approximately the same time, that the spectral data was generated.

A computing device or system such as training server 112 then trains 404 the deep learning model (e.g., CNN 300) using the spectral data of the historical data set 402 as features/inputs, and using the corresponding analytical measurements as labels, to produce a trained deep learning model 406. In run-time operation, deep learning model 406 operates on spectral data 408 (e.g., Raman scan vectors generated by Raman analyzer 106 and Raman probe 108, or other 1D spectral data generated by a different type of spectroscopy system) to generate predicted output 410 (e.g., a predicted metabolite concentration).

While not shown in FIG. 4, pre-processing of the spectral data occurs both at the training 404 stage (at any point prior to inputting the Raman scan vector or other spectral data into the model being trained), and when the deep learning model 406 is used during run-time to generate the predicted output 410 (e.g., shortly after the Raman analyzer 106 generates a Raman scan vector). This pre-processing includes converting the spectral data from its original 1D format into a pseudo-image (i.e., a 2D spectral data matrix) so that the model can process the spectral data in essentially the same manner that the model would process an image.

Each Raman (or NIR, etc.) spectroscopic measurement, when converted to a pseudo-image, may become a relatively large input image with high x and y dimensions. Feeding such an image directly into a machine learning model can require that the model have a large number of parameters, which may unnecessarily increase computational time. Therefore, one or more steps of pre-processing and dimension reduction may be applied to the Raman scan vector (or other 1D spectral data) before prediction application 132 applies that data as a model input.

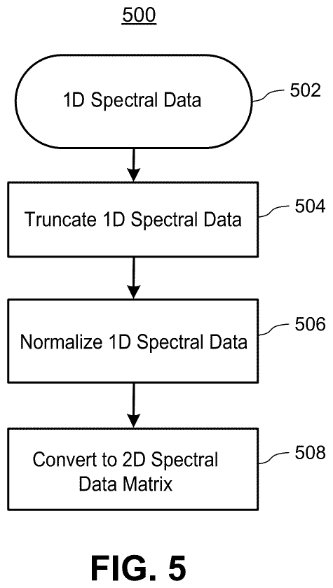

FIG. 5 depicts example pre-processing 500 of 1D spectral data 502 (e.g., a Raman scan vector) that may be implemented in system 100 of FIG. 1, to prepare 1D spectral data 502 for processing by a deep learning model such as DL model 130, CNN 300, or deep learning model 406. Pre-processing 500 may occur both during training and during run-time operation, to ensure that model inputs have a consistent format at both stages. In some embodiments, pre-processing 500 is performed by prediction application 132, or by data cleaning software 134.

In the depicted embodiment, pre-processing 500 includes truncating 504 the 1D spectral data 502. The truncating 504 may include removing spectral data points (e.g., spectral data points corresponding to particular wave numbers of a Raman scan) that are known (e.g., via earlier experimentation) to be less correlated with the model outputs (i.e., have less predictive power). In some embodiments, truncating 504 includes removing spectral data points corresponding to one or more contiguous sequences of wave numbers. For example, for Raman scan vectors with wave numbers from 100 to 3425, truncating 504 may include removing (e.g., ignoring or otherwise not using) the spectral data points corresponding to all wave numbers outside the range from 450 to 1893. For example, the remaining range of 450 to 1893 may be particularly well-suited for predicting metabolite concentrations.

In other embodiments, truncating 504 also, or instead, includes removing a non-contiguous sequence of spectral data points. For example, for Raman scan vectors with wave numbers from 100 to 3425, truncating 504 may include removing (e.g., ignoring or otherwise not using) the spectral data points corresponding to all wave numbers outside the range from 500 to 3199, and then further removing X of every Y remaining data points (e.g., two of every three data points) in repeating fashion (e.g., keep, remove, remove, keep, remove, remove, etc.). Removing the spectral data points corresponding to wave numbers 100 to 499 can be beneficial because that range has been found to suffer from interference from the Raman instrument. Removing the spectral data points corresponding to wave numbers 3200 to 3325 can be beneficial because that range has been found to exhibit relatively high variability.

After truncating 504 the 1D spectral data 502, the remaining 1D spectral data is normalized 506. Normalization 506 may include normalizing the intensity values across the remaining spectral (e.g., wave number) range of the truncated 1D spectral data. For example, normalization 506 may include mapping the truncated 1D spectral data to a standard distribution with zero mean and unity standard deviation. As another example, normalization 506 may include mapping the minimum and maximum values (e.g., intensity levels) of the 1D spectral data to −1 and +1, respectively.

The truncated, normalized 1D spectral data is then converted 508 (reshaped) from its original 1D format into a 2D matrix of suitable size. For the above example in which the Raman scan vector was truncated 504 down to only wave numbers 450 to 1893 (resulting in 1444 total data points), the 2D spectral data matrix may be a 38×38 matrix. For another example in which the Raman scan vector was truncated 504 down to only wave numbers 500 to 3199 and then two of every three remaining wave numbers were removed (resulting in 900 total spectral data points), the 2D spectral data matrix may be a 30×30 matrix.

After the conversion 508, the prediction application 132 inputs the 2D spectral data matrix into the DL model 130. It is understood that, in some embodiments, pre-processing 500 includes additional and/or different steps than are shown in FIG. 5.

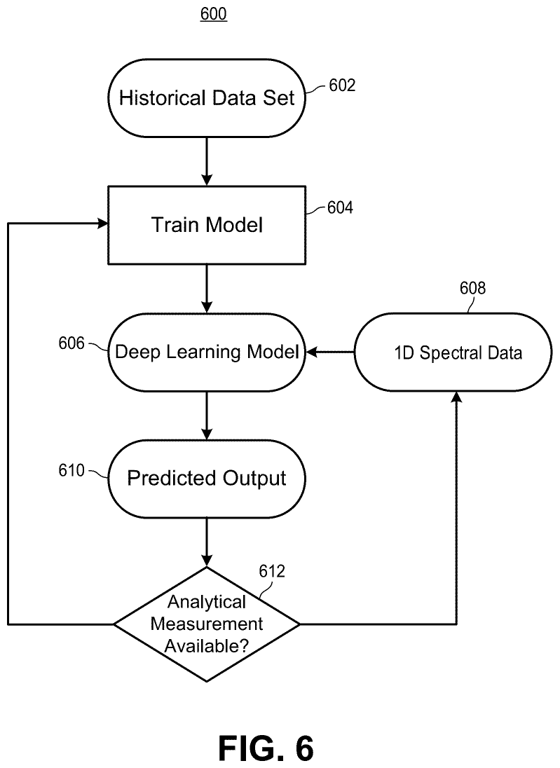

As noted above, the techniques described herein may make recalibration, or at least frequent recalibration, unnecessary. In some embodiments, however, computer 110 or training server 112 does recalibrate DL model 130 from time to time. FIG. 6 depicts one such embodiment, in an example data flow 600 that may occur in system 100 of FIG. 1 or system 200 of FIG. 2. In data flow 600, a historical data set 602 may reside in training database 138. The historical data set 602 includes 1D spectral data (e.g., Raman scan vectors) generated by suitable devices/systems (e.g., similar to Raman analyzer 106 and Raman probe 108), and corresponding labels. The labels may be actual measurements of the parameter (e.g., metabolite level) of interest, taken by an analytical instrument (similar to instrument(s) 104) at the same time, or approximately the same time, that the 1D spectral data was generated.

A computing device or system such as training server 112 then trains 604 the deep learning model (e.g., CNN 300) using the 1D spectral data of the historical data set 602 as features/inputs, and using the corresponding analytical measurements as labels, to produce a trained deep learning model 606. During run-time operation, deep learning model 606 operates on 1D spectral data 608 (e.g., Raman scan vectors generated by Raman analyzer 106 and Raman probe 108) to generate predicted output 610 (e.g., a predicted metabolite concentration). While not shown in FIG. 6, pre-processing of the 1D spectral data (e.g., similar to pre-processing 500) may occur both at the training 604 stage (at any point prior to inputting the Raman scan vector or other 1D spectral data into the model being trained), and when the deep learning model 606 is used during run-time to generate the predicted output 610 (e.g., shortly after the Raman analyzer 106 generates a Raman scan vector).

Also in data flow 600, computer 110 or training server 112 may determine 612 whether an analytical measurement corresponding to the most recent Raman scan vector or other spectral data is available (e.g., from analytical instrument(s) 104). If so, computer 110 or training server 112 uses the new measurement as a label (and the corresponding spectral data as a model feature/input) to further train (i.e., tune) the deep learning model 606. If no such measurement is available, the model is not further trained/tuned.

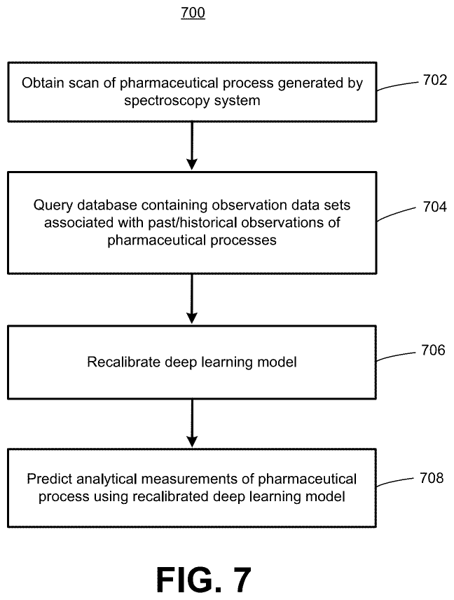

In some embodiments, the techniques described herein (e.g., pre-processing 500) are used in combination with JITL. The example method 700 of FIG. 7 depicts one such embodiment. The method 700 may be performed by computer 110 (e.g., processing unit 120 executing instructions stored in memory 128) and/or training server 112, for example. In the method 700, at block 702, a new scan of a pharmaceutical process is obtained. The scan comprises 1D spectral data (e.g., intensity values ordered according to wave number, or a sequence of [wave number, intensity] tuples) that was generated by a spectroscopy system (e.g., a Raman scan vector generated by Raman analyzer 106 using Raman probe 108), and may be a single raw scan, an aggregation of multiple scans, an average of multiple scans, and so on.

At block 704, a database containing observation data sets (e.g., similar to training database 138) is queried. The observation data sets are associated with past/historical observations of pharmaceutical processes (e.g., the same type of pharmaceutical process referenced above in connection with block 702). Each of the observation data sets may include, in addition to a scan (e.g., a Raman scan vector or other 1D spectral data), a corresponding analytical measurement. The analytical measurement may be a media component concentration, media state (e.g., glucose, lactate, glutamate, glutamine, ammonia, amino acids, Na+, K+ and other nutrients or metabolites, pH, pCO₂, pO₂, osmolality, etc.), viable cell density, titer, a critical quality attribute, and/or cell state, for example.

Block 704 includes determining a query point based at least in part on the new 1D spectral data. Depending on the embodiment, the query point may be determined based on the raw 1D spectral data, or after suitable pre-processing of the raw 1D spectral data (e.g., similar to pre-processing 500). In some embodiments, the query point is also determined based on other information, such as a media profile associated with a biopharmaceutical process (e.g., a fluid type, specific nutrients, a pH level, etc.), and/or one or more operating conditions under which a biopharmaceutical process is analyzed (e.g., a metabolite concentration set point, etc.), for example. Block 704 may then include selecting as training data, from among the observation data sets, those observation data sets that satisfy one or more relevancy criteria with respect to the query point. If the query point included a Raman spectral scan vector, for example, block 704 may include comparing that Raman spectral scan vector to the spectral scan vectors associated with each of the past observations represented in the observation database.

At block 706, the deep learning model (e.g., DL model 130, CNN 300, or deep learning model 406) is recalibrated (retrained) using the portion of the observation data sets that were selected at block 704 in response to the query. At block 708, characteristics or parameters of the pharmaceutical process are predicted by the recalibrated deep learning model operating on additional 1D spectral data (e.g., Raman scan vectors newly generated by Raman analyzer 106), after that additional 1D spectral data has been pre-processed (e.g., according to pre-processing 500).

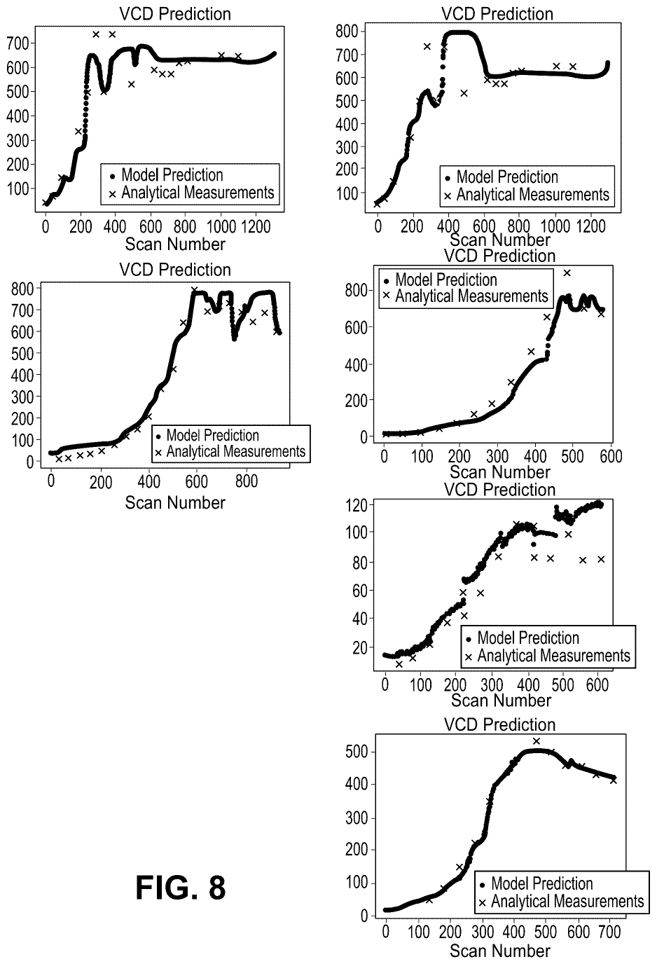

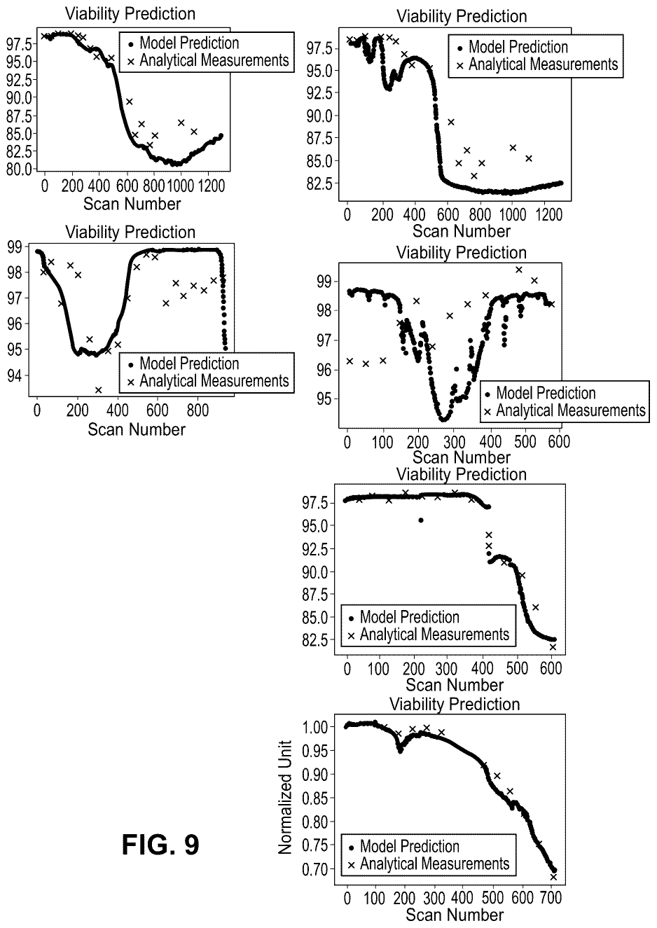

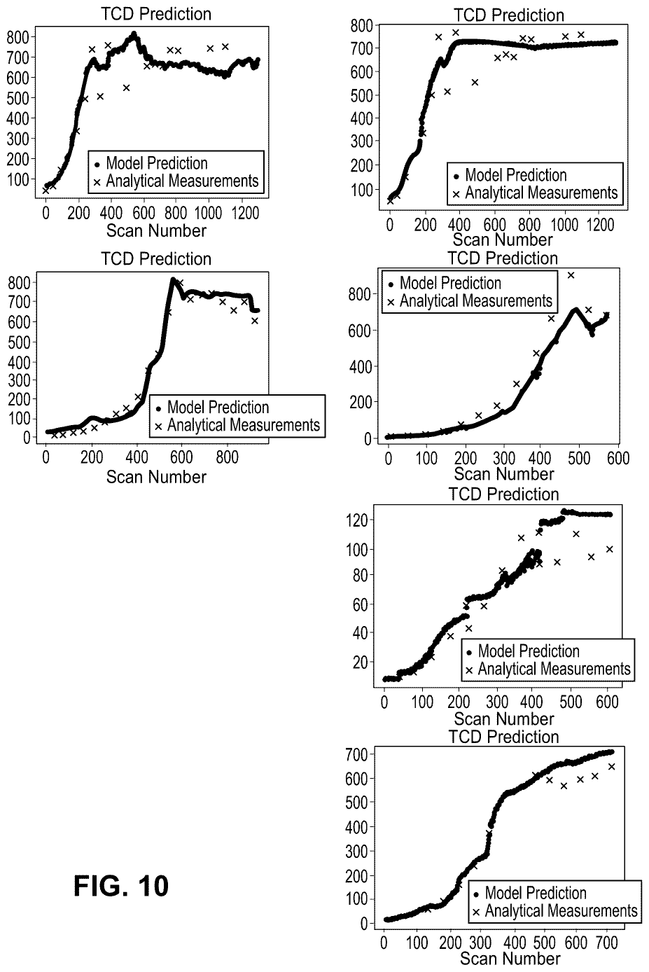

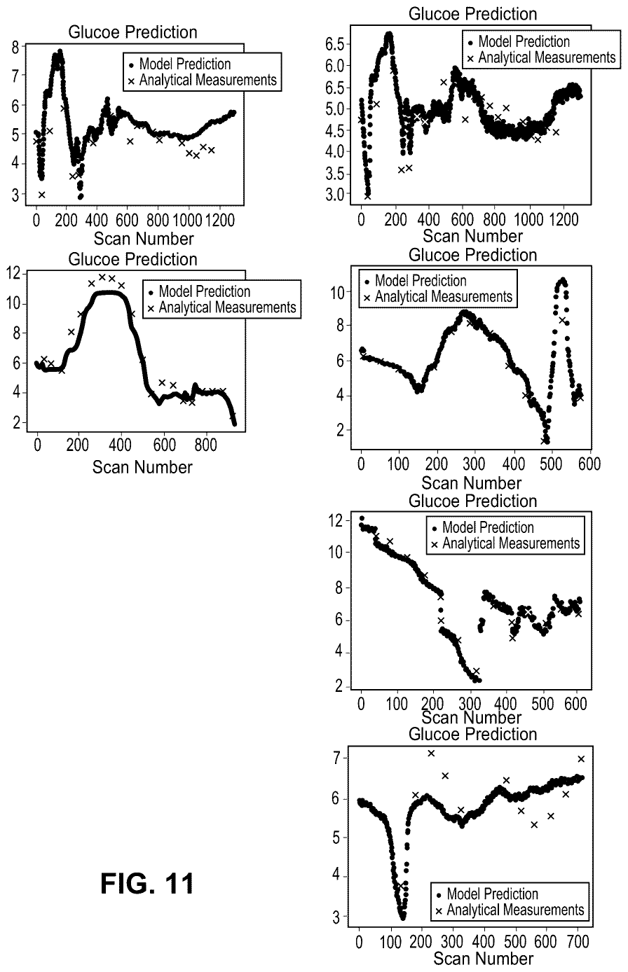

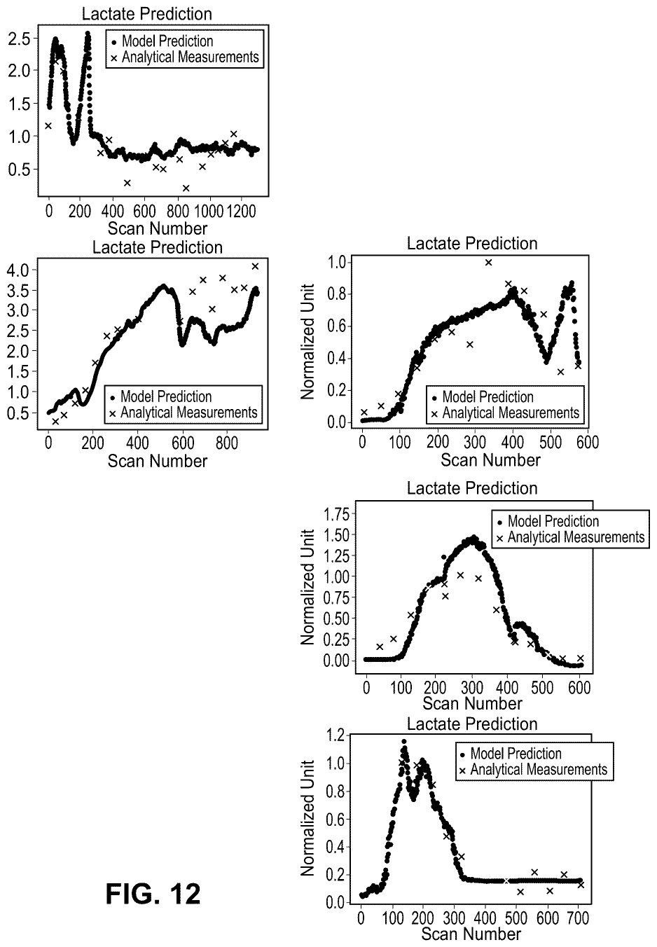

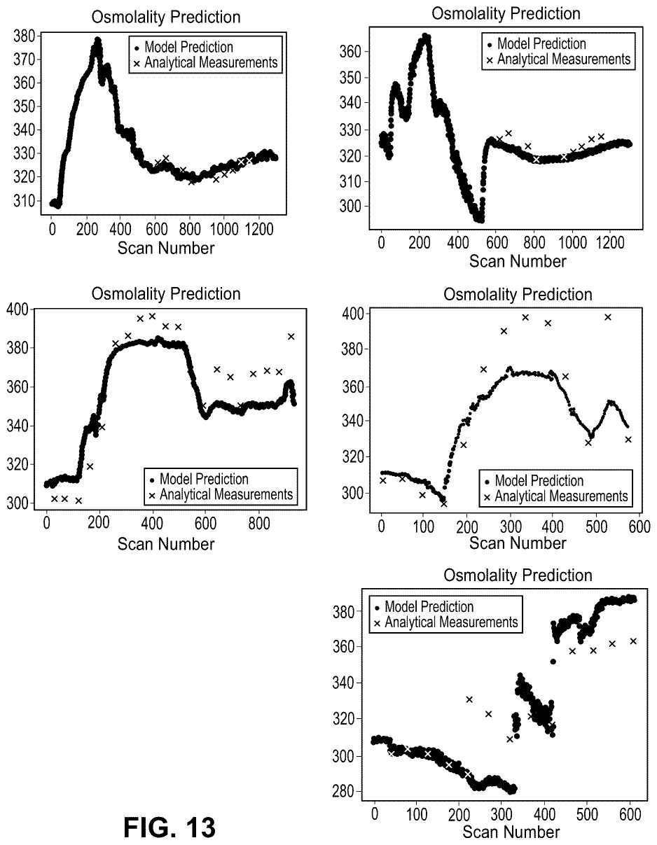

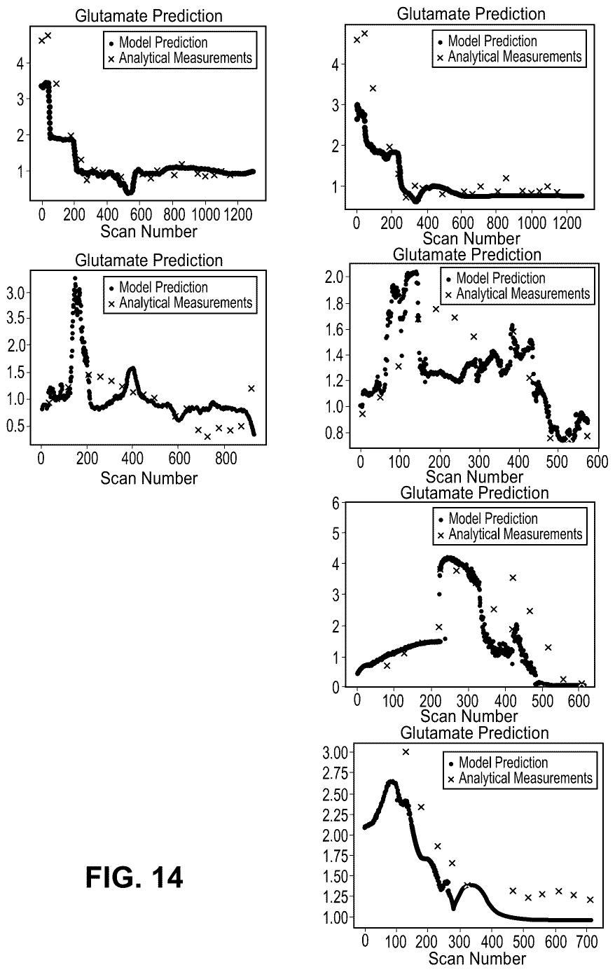

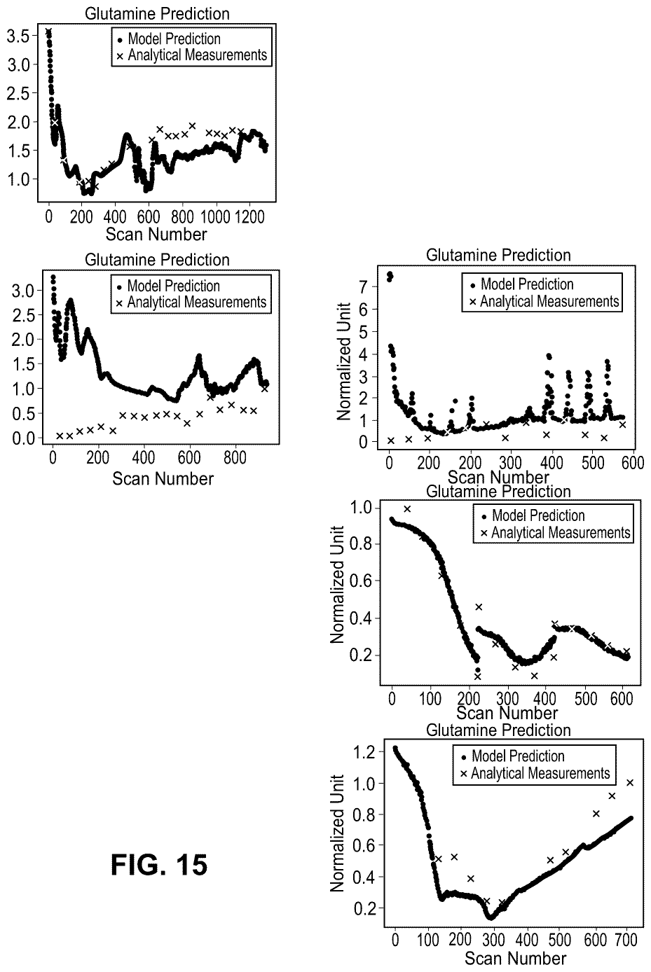

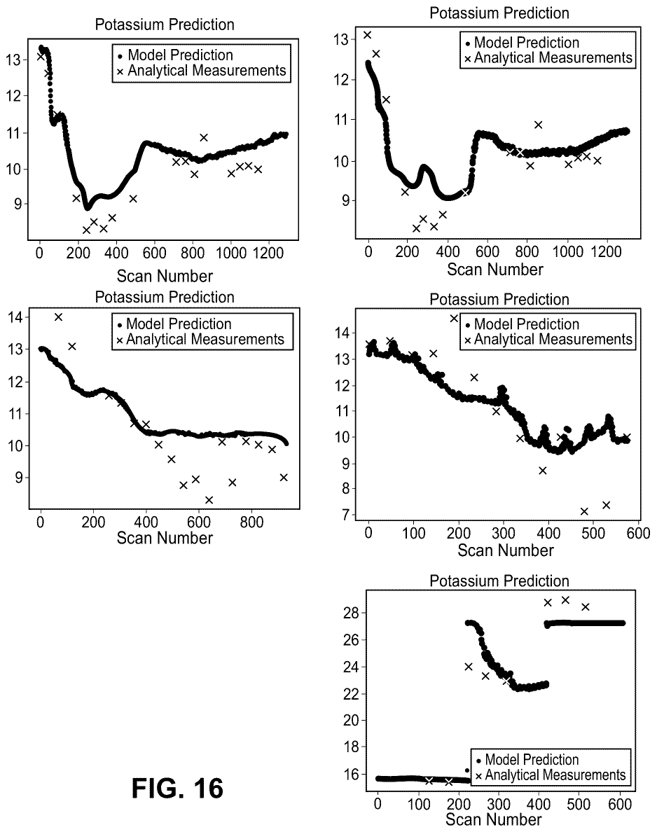

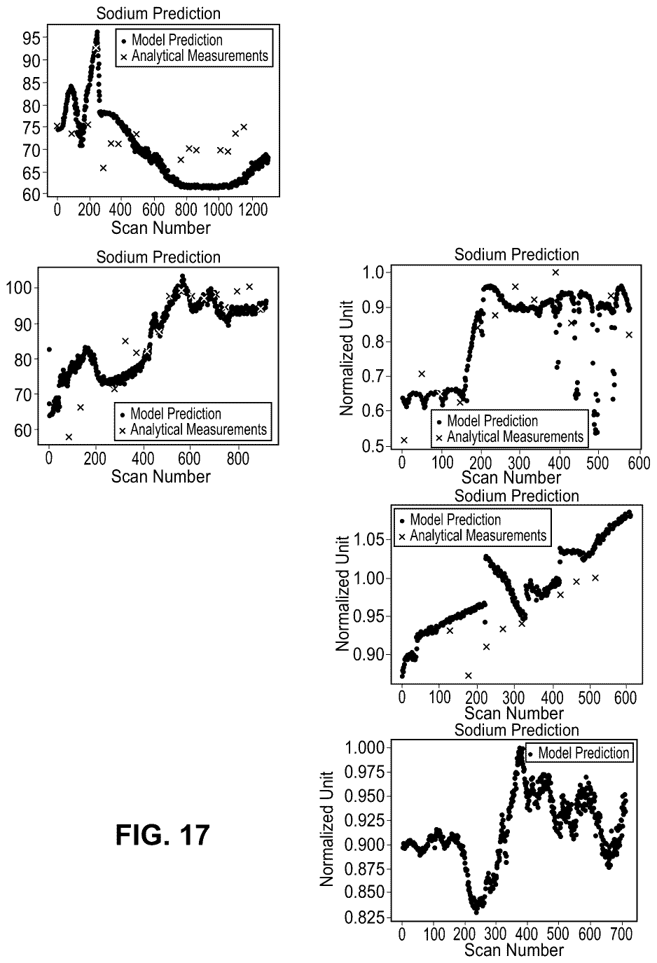

FIGS. 8-17 depict experimental results for various parameters (VCD, viability, TCD, glucose concentration, lactate concentration, osmolality, glutamate concentration, glutamine concentration, potassium concentration, and sodium concentration, respectively) and an example implementation of a deep learning model (in these examples, a CNN model similar to CNN model 300). In the plots of FIGS. 8-17, each “x” symbol represents an actual measurement of the parameter/attribute being measured (e.g., generated by an analytical instrument similar to one of analytical instrument(s) 104 of FIG. 1 or FIG. 2), while the solid lines represent the predicted values of the parameter/attribute (as predicted by the CNN model). In each of FIGS. 8-17, the plots in the left-hand column represent results obtained using a first method of pre-processing, while the plots in the right-hand column represent results obtained using a second method of pre-processing. The “first method” is the pre-processing 500 of FIG. 5, with the 1D spectral data (here, a Raman scan vector) being truncated down to the wave numbers starting at 450 and ending at 1893, and with the 2D spectral data matrix being a 38×38 matrix. The “second method” is also the pre-processing 500 of FIG. 5, but with the 1D spectral data (again, a Raman scan vector) being truncated down to only the first of every three wave numbers in the range 500 to 3199, and with the 2D spectral data matrix being a 30×30 matrix. In each of FIGS. 8-17, each row of plots corresponds to a different drug product. In FIG. 8, for example, results are shown for a first and second drug product for both the first and the second method or pre-processing, but results for a third and fourth drug product are only shown for the second method of pre-processing.

As seen in FIGS. 8-17, when using the first method of pre-processing, the predicted values for VCD, viability, and glucose were generally in close agreement with the analytical measurements. However, the predicted values for osmolality, glutamine, potassium, and sodium were less consistent. When the second method of pre-processing was applied, the predicted values for all attributes was generally more consistent than was seen with the first method. Depending on the metabolite being measured, it may be preferable to use one pre-processing method over the other.

Additional considerations pertaining to this disclosure will now be addressed.

The terms “polypeptide” or “protein” are used interchangeably throughout and refer to a molecule comprising two or more amino acid residues joined to each other by peptide bonds. Polypeptides and proteins also include macromolecules having one or more deletions from, insertions to, and/or substitutions of the amino acid residues of the native sequence, that is, a polypeptide or protein produced by a naturally-occurring and non-recombinant cell; or is produced by a genetically-engineered or recombinant cell, and comprise molecules having one or more deletions from, insertions to, and/or substitutions of the amino acid residues of the amino acid sequence of the native protein. Polypeptides and proteins also include amino acid polymers in which one or more amino acids are chemical analogs of a corresponding naturally-occurring amino acid and polymers. Polypeptides and proteins are also inclusive of modifications including, but not limited to, glycosylation, lipid attachment, sulfation, gamma-carboxylation of glutamic acid residues, hydroxylation and ADP-ribosylation.

Polypeptides and proteins can be of scientific or commercial interest, including protein-based therapeutics. Proteins include, among other things, secreted proteins, non-secreted proteins, intracellular proteins or membrane-bound proteins. Polypeptides and proteins can be produced by recombinant animal cell lines using cell culture methods and may be referred to as “recombinant proteins”. The expressed protein(s) may be produced intracellularly or secreted into the culture medium from which it can be recovered and/or collected. Proteins include proteins that exert a therapeutic effect by binding a target, particularly a target among those listed below, including targets derived therefrom, targets related thereto, and modifications thereof.

Proteins “antigen-binding proteins”. Antigen-binding protein refers to proteins or polypeptides that comprise an antigen-binding region or antigen-binding portion that has a strong affinity for another molecule to which it binds (antigen). Antigen-binding proteins encompass antibodies, peptibodies, antibody fragments, antibody derivatives, antibody analogs, fusion proteins (including single-chain variable fragments (scFvs) and double-chain (divalent) scFvs, muteins, xMAbs, and chimeric antigen receptors (CARs).

An scFv is a single chain antibody fragment having the variable regions of the heavy and light chains of an antibody linked together. See U.S. Pat. Nos. 7,741,465, and 6,319,494 as well as Eshhar et al., Cancer Immunol Immunotherapy (1997) 45:131-136. An scFv retains the parent antibody's ability to specifically interact with target antigen.

The term “antibody” includes reference to both glycosylated and non-glycosylated immunoglobulins of any isotype or subclass or to an antigen-binding region thereof that competes with the intact antibody for specific binding. Unless otherwise specified, antibodies include human, humanized, chimeric, multi-specific, monoclonal, polyclonal, heteroIgG, XmAbs, bispecific, and oligomers or antigen binding fragments thereof. Antibodies include the IgG1-, IgG2-IgG3- or IgG4-type. Also included are proteins having an antigen binding fragment or region such as Fab, Fab′, F(ab′)2, Fv, diabodies, Fd, dAb, maxibodies, single chain antibody molecules, single domain VHH, complementarity determining region (CDR) fragments, scFv, diabodies, triabodies, tetrabodies and polypeptides that contain at least a portion of an immunoglobulin that is sufficient to confer specific antigen binding to a target polypeptide.

Also included are human, humanized, and other antigen-binding proteins, such as human and humanized antibodies, that do not engender significantly deleterious immune responses when administered to a human.

Also included are peptibodies, polypeptides comprising one or more bioactive peptides joined together, optionally via linkers, with an Fc domain. See U.S. Pat. Nos. 6,660,843, 7,138,370 and 7,511,012.

Proteins also include genetically engineered receptors such as chimeric antigen receptors (CARs or CAR-Ts) and T cell receptors (TCRs). CARs typically incorporate an antigen binding domain (such as scFv) in tandem with one or more costimulatory (“signaling”) domains and one or more activating domains.

Also included are bispecific T cell engagers (BiTE®) antibody constructs are recombinant protein constructs made from two flexibly linked antibody derived binding domains (see WO 99/54440 and WO 2005/040220). One binding domain of the construct is specific for a selected tumor-associated surface antigen on target cells; the second binding domain is specific for CD3, a subunit of the T cell receptor complex on T cells. The BiTER constructs may also include the ability to bind to a context independent epitope at the N-terminus of the CD3s chain (WO 2008/119567) to more specifically activate T cells. Half-life extended BiTE® constructs include fusion of the small bispecific antibody construct to larger proteins, which preferably do not interfere with the therapeutic effect of the BiTE® antibody construct. Examples for such further developments of bispecific T cell engagers comprise bispecific Fc-molecules e.g. described in US 2014/0302037, US 2014/0308285, WO 2014/151910 and WO 2015/048272. An alternative strategy is the use of human serum albumin (HAS) fused to the bispecific molecule or the mere fusion of human albumin binding peptides (see e.g. WO 2013/128027, WO2014/140358). Another HLE BiTE® strategy comprises fusing a first domain binding to a target cell surface antigen, a second domain binding to an extracellular epitope of the human and/or the Macaca CD3e chain and a third domain, which is the specific Fc modality (WO 2017/134140).

In some embodiments, proteins may include colony stimulating factors, such as granulocyte colony-stimulating factor (G-CSF). Such G-CSF agents include, but are not limited to, Neupogen® (filgrastim) and Neulasta® (pegfilgrastim). Also included are erythropoiesis stimulating agents (ESA), such as Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, and epoetin delta, epoetin omega, epoetin iota, tissue plasminogen activator, GLP-1 receptor agonists, as well as the molecules or variants or analogs thereof and biosimilars of any of the foregoing.

In some embodiments, proteins may include proteins that bind specifically to one or more CD proteins, HER receptor family proteins, cell adhesion molecules, growth factors, nerve growth factors, fibroblast growth factors, transforming growth factors (TGF), insulin-like growth factors, osteoinductive factors, insulin and insulin-related proteins, coagulation and coagulation-related proteins, colony stimulating factors (CSFs), other blood and serum proteins blood group antigens; receptors, receptor-associated proteins, growth hormones, growth hormone receptors, T-cell receptors; neurotrophic factors, neurotrophins, relaxins, interferons, interleukins, viral antigens, lipoproteins, integrins, rheumatoid factors, immunotoxins, surface membrane proteins, transport proteins, homing receptors, addressins, regulatory proteins, and immunoadhesins.

In some embodiments proteins may include proteins that bind to one of more of the following, alone or in any combination: CD proteins including but not limited to CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des (1-3)-IGF-I (brain IGF-I), and osteoinductive factors, insulins and insulin-related proteins, including but not limited to insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; (x) neurotrophic factors, including but not limited to, bone-derived neurotrophic factor (BDNF) and neurotrophin-3, -4, -5, or -6 (NT-3, NT-4, NT-5, or NT-6); (xi) relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP; (xiv) viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide, Dnase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, eotaxin-1, MUC1, CEA, c-MET, Claudin-18, GPC-3, EPHA2, FPA, LMP1, MG7, NY-ESO-1, PSCA, ganglioside GD2, glanglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL1, mannose receptor/hCGB, hepatitis-C virus, mesothelin dsFv [PE38 conjugate, Legionella pneumophila (Ily), IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40L, α4β7, platelet specific (platelet glycoprotein IIb/IIIb (PAC-1), transforming growth factor beta (TFGβ), Zona pellucida sperm-binding protein 3 (ZP-3), TWEAK, platelet derived growth factor receptor alpha (PDGFRα), sclerostin, and biologically active fragments or variants of any of the foregoing.

In another embodiment, proteins include abciximab, adalimumab, adecatumumab, aflibercept, alemtuzumab, alirocumab, anakinra, atacicept, basiliximab, belimumab, bevacizumab, biosozumab, blinatumomab, brentuximab vedotin, brodalumab, cantuzumab mertansine, canakinumab, cetuximab, certolizumab pegol, conatumumab, daclizumab, denosumab, eculizumab, edrecolomab, efalizumab, epratuzumab, etanercept, evolocumab, galiximab, ganitumab, gemtuzumab, golimumab, ibritumomab tiuxetan, infliximab, ipilimumab, lerdelimumab, lumiliximab, Ixdkizumab, mapatumumab, motesanib diphosphate, muromonab-CD3, natalizumab, nesiritide, nimotuzumab, nivolumab, ocrelizumab, ofatumumab, omalizumab, oprelvekin, palivizumab, panitumumab, pembrolizumab, pertuzumab, pexelizumab, ranibizumab, rilotumumab, rituximab, romiplostim, romosozumab, sargamostim, tocilizumab, tositumomab, trastuzumab, ustekinumab, vedolizumab, visilizumab, volociximab, zanolimumab, zalutumumab, and biosimilars of any of the foregoing.

Proteins encompass all of the foregoing and further include antibodies comprising 1, 2, 3, 4, 5, or 6 of the complementarity determining regions (CDRs) of any of the aforementioned antibodies. Also included are variants that comprise a region that is 70% or more, especially 80% or more, more especially 90% or more, yet more especially 95% or more, particularly 97% or more, more particularly 98% or more, yet more particularly 99% or more identical in amino acid sequence to a reference amino acid sequence of a protein of interest. Identity in this regard can be determined using a variety of well-known and readily available amino acid sequence analysis software. Preferred software includes those that implement the Smith-Waterman algorithms, considered a satisfactory solution to the problem of searching and aligning sequences. Other algorithms also may be employed, particularly where speed is an important consideration. Commonly employed programs for alignment and homology matching of DNAs, RNAs, and polypeptides that can be used in this regard include FASTA, TFASTA, BLASTN, BLASTP, BLASTX, TBLASTN, PROSRCH, BLAZE, and MPSRCH, the latter being an implementation of the Smith-Waterman algorithm for execution on massively parallel processors made by MasPar.

Some of the figures described herein illustrate example block diagrams having one or more functional components. It will be understood that such block diagrams are for illustrative purposes and the devices described and shown may have additional, fewer, or alternate components than those illustrated. Additionally, in various embodiments, the components (as well as the functionality provided by the respective components) may be associated with or otherwise integrated as part of any suitable components.

Embodiments of the disclosure relate to a non-transitory computer-readable storage medium having computer code thereon for performing various computer-implemented operations. The term “computer-readable storage medium” is used herein to include any medium that is capable of storing or encoding a sequence of instructions or computer codes for performing the operations, methodologies, and techniques described herein. The media and computer code may be those specially designed and constructed for the purposes of the embodiments of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as ASICs, programmable logic devices (“PLDs”), and ROM and RAM devices.

Examples of computer code include machine code, such as produced by a compiler, and files containing higher-level code that are executed by a computer using an interpreter or a compiler. For example, an embodiment of the disclosure may be implemented using Java, C++, Python, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

As used herein, the singular terms “a,” “an,” and “the” may include plural referents, unless the context clearly dictates otherwise.

As used herein, the terms “connect,” “connected,” and “connection” refer to an operational coupling or linking. Connected components can be directly or indirectly coupled to one another, for example, through another set of components.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific embodiments thereof, these descriptions and illustrations do not limit the present disclosure. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes, tolerances and/or other reasons. There may be other embodiments of the present disclosure which are not specifically illustrated. The specification (other than the claims) and drawings are to be regarded as illustrative rather than restrictive. Modifications may be made to adapt a particular situation, material, composition of matter, technique, or process to the objective, spirit and scope of the present disclosure. All such modifications are intended to be within the scope of the claims appended hereto. While the techniques disclosed herein have been described with reference to particular operations performed in a particular order, it will be understood that these operations may be combined, sub-divided, or re-ordered to form an equivalent technique without departing from the teachings of the present disclosure. Accordingly, unless specifically indicated herein, the order and grouping of the operations are not limitations of the present disclosure.

1. A computer-implemented method for monitoring and/or controlling a pharmaceutical process, the method comprising:
obtaining, by one or more processors, one-dimensional (1D) spectral data generated by a spectroscopy system when scanning the pharmaceutical process;
converting, by the one or more processors, the 1D spectral data to a two-dimensional (2D) spectral data matrix; and
predicting, by the one or more processors, a parameter of the pharmaceutical process, wherein predicting the parameter of the pharmaceutical process includes applying the 2D spectral data matrix to an input layer of a deep learning model.

2. The computer-implemented method of claim 1, wherein the 1D spectral data comprises (i) a sequence of tuples each comprising an intensity value and a corresponding wave number, or (ii) a sequence of intensity values in which each position corresponds to a respective wave number.

3. The computer-implemented method of claim 1, wherein the spectroscopy system is a Raman spectroscopy system, a near infrared (NIR) spectroscopy system, a high performance liquid chromatography (HPLC) spectroscopy system, an ultra high performance liquid chromatography (UPLC) spectroscopy system, or a mass spectrometry system.

4. The computer-implemented method of claim 1, wherein the deep learning model is a convolutional neural network (CNN) model.

5. The computer-implemented method of claim 1, wherein converting the 1D spectral data to the 2D spectral data matrix includes:
truncating the 1D spectral data by removing a plurality of spectral data points; and
using the truncated 1D spectral data to populate the 2D spectral data matrix.

6. The computer-implemented method of claim 5, wherein converting the 1D spectral data to the 2D spectral data matrix further includes:
before or after truncating the 1D spectral data, normalizing the 1D spectral data.

7. The computer-implemented method of claim 5, wherein truncating the 1D spectral data includes removing spectral data points that are less correlated with the parameter.

8. The computer-implemented method of claim 5, wherein truncating the 1D spectral data includes removing spectral data points in one or more predetermined ranges of spectral data points.

9. The computer-implemented method of claim 8, wherein removing spectral data points in the one or more predetermined ranges of spectral data points includes one or both of:
removing spectral data points in one or more ranges of spectral data points known to have high variability; and
removing spectral data points in one or more ranges of spectral data points known to exhibit spectroscopy system interference.

10. The computer-implemented method of claim 5, wherein truncating the 1D spectral data includes removing X of every Y spectral data points in a predetermined range of spectral data points, with X and Y being predetermined positive integers and with Y being greater than X.

11. The computer-implemented method of claim 10, wherein X equals 2 and Y equals 3.

12. The computer-implemented method of claim 1, further comprising:
controlling, by the one or more processors and based at least in part on the predicted parameter of the pharmaceutical process, at least one parameter of the pharmaceutical process.

13. The computer-implemented method of claim 1, further comprising:
causing, by the one or more processors, the predicted parameter to be presented to a user via a display.

14. The computer-implemented method of claim 1, wherein the predicted parameter of the pharmaceutical process is a media component concentration, a media state, a viable cell density, a titer, a critical quality attribute, or a cell state.

15. The computer-implemented method of claim 1, wherein the predicted parameter of the pharmaceutical process is a concentration of glucose, lactate, glutamate, glutamine, ammonia, amino acids, Na⁺, or K⁺.

16. The computer-implemented method of claim 1, wherein the predicted parameter of the pharmaceutical process is pH, pCO₂, pO₂, or osmolality.

17. The computer-implemented method of claim 1, further comprising, before obtaining the 1D spectral data:
training the deep learning model using historical 1D spectral data generated by one or more spectroscopy systems and corresponding actual analytical measurements of pharmaceutical processes.

18. The computer-implemented method of claim 1, further comprising:
obtaining, by an analytical instrument, an actual analytical measurement of the pharmaceutical process; and
training the deep learning model using (i) additional 1D spectral data that the spectroscopy system generated when the actual analytical measurement was obtained, and (ii) the actual analytical measurement of the pharmaceutical process.

19. The computer-implemented method of claim 1, further comprising:
determining, by the one or more processors, a query point associated with scanning of the pharmaceutical process by the spectroscopy system;
querying, by the one or more processors, a database containing a plurality of observation data sets associated with past observations of pharmaceutical processes, wherein each of the observation data sets includes associated 1D spectral data and a corresponding actual analytical measurement, and wherein querying the database includes selecting as training data, from among the plurality of observation data sets, observation data sets that satisfy one or more relevancy criteria with respect to the query point; and
training, by the one or more processors and using the selected training data, the deep learning model using the observation data sets that satisfy the one or more relevancy criteria with respect to the query point.

20. The computer-implemented method of claim 19, wherein:
determining the query point includes determining the query point based at least in part on new 1D spectral data, the new 1D spectral data being generated by the spectroscopy system when scanning the pharmaceutical process; and
selecting as training data the observation data sets that satisfy the one or more relevancy criteria with respect to the query point includes comparing the new 1D spectral data on which determination of the query point was based to 1D spectral data associated with the past observations of the pharmaceutical processes.

21. The computer-implemented method of claim 19, wherein determining the query point includes:
determining the query point based at least in part on one or both of (i) a media profile associated with the pharmaceutical process, and (ii) one or more operating conditions under which the pharmaceutical process is analyzed.

22. The computer-implemented method of claim 1, wherein the pharmaceutical process is a cell culture process.

23. One or more non-transitory computer-readable media storing instructions for monitoring and/or controlling a pharmaceutical process, wherein the instructions, when executed by one or more processors, cause the one or more processors to:
obtain one-dimensional (1D) spectral data generated by a spectroscopy system when scanning the pharmaceutical process;
convert the 1D spectral data to a two-dimensional (2D) spectral data matrix; and
predict a parameter of the pharmaceutical process, wherein predicting the parameter of the pharmaceutical process includes applying the 2D spectral data matrix to an input layer of a deep learning model.
